(19) United States
(12) Patent Application Publication
(10) Pub. No.: US 2025/0381140 A1
(43) Pub. Date: Dec. 18, 2025

(54) LIPID NANOPARTICLES USING CATIONIC CHOLESTEROL FOR LOCAL DELIVERY FOR NUCLEIC ACID DELIVERY

(71) Applicant: GREEN CROSS CORPORATION, Gyeonggi-do (KR)

(72) Inventors: Joon Young PARK, Gyeonggi-do (KR); Jiyeon SON, Gyeonggi-do (KR); Hyo Jung NAM, Gyeonggi-do (KR); Jung-Eun LEE, Gyeonggi-do (KR)

(21) Appl. No.: 18/878,063

(22) PCT Filed: Jul. 4, 2023
(86) PCT No.: PCT/KR2023/009400

§ 371 Date: Dec. 22, 2024

(30) Foreign Application Priority Data
Jul. 6, 2022 (KR) 10-2022-0083137

(51) Int. Cl. A61K 9/1272; A61K 9/51; A61K 31/7105; A61K 39/00; A61K 39/145; B82Y 5/00; C12N 7/00

(52) U.S. Cl. CPC A61K 9/1272; A61K 9/5123; A61K 31/7105; A61K 39/145; C12N 7/00; A61K 2039/55555; A61K 2039/575; B82Y 5/00; C12N 2760/16134

(57) ABSTRACT

The present invention is directed to lipid nanoparticles using cationic cholesterol for topical delivery for nucleic acid delivery, and when administered locally, side effects caused by systemic drug delivery can be minimized and protein expression can be confined to the site of administration. In addition, the duration of protein expression at the site of administration can be increased, and thus the lipid nanoparticles can be useful in the technical field related to nucleic acid therapeutics.

### TECHNICAL FIELD

The present invention relates to lipid nanoparticles for nucleic acid delivery, and more particularly to lipid nanoparticles for topical delivery comprising cationic cholesterol, a lipid nanoparticle composition comprising the nanoparticles and a nucleic acid, and a method of preventing or treating a disease using the same.

### Background Art

With the recent emergence of COVID-19 vaccines, the expectations and importance of mRNA-based vaccines are increasing. Both licensed vaccines (Spikevax™, Comirnaty™) use lipid nanoparticles (LNPs) as delivery carriers, and efforts are underway to continuously improve efficacy through optimization of LNP compositions and development of additional ionizable lipids.

In general, however, intramuscularly injected LNPs spread throughout the body as well as the injected muscle site through lymphatic vessels and blood, which raises the risk of systemic reaction. In particular, for COVID-19 vaccines, the possibility of causing myocarditis, which is an inflammation of the heart muscle, is raised due to some heart-delivered vaccines. Accordingly, when developing prophylactic vaccines for general healthy people after the end of the pandemic, delivery carriers with improved safety profile are required.

With the goal of addressing these limitations, in the present invention, attempts have been made to develop a delivery carrier that reduces systemic reaction by expressing mRNA only at the site of injection and minimizing systemic distribution. In the future, such a delivery carrier is expected to be applicable to topical administration such as intratumoral injection, intradermal injection, intracerebral injection, etc., in addition to intramuscular injection. It is expected that the administration dose can be fully utilized by confining MRNA expression to the site of administration and reducing the amount lost outside the site of administration.

Furthermore, it was confirmed that the present invention is capable of increasing the duration of protein expression at the muscle site. Typically, a decrease in protein expression at the muscle site after intramuscular injection shows zero-order kinetics, and it appeared that the present invention is capable of remarkably increasing the duration of protein expression. Thereby, it is expected that the therapeutic dose may be reduced.

Taken together, the present invention pertains to lipid nanoparticles comprising cationic cholesterol and a composition comprising the same, and particularly to a delivery carrier capable of 1) increasing safety by confining protein expression to a topical site and 2) increasing the duration of protein expression.

The above information described in the background section is only for improving the understanding of the background of the present invention, and it may not include information forming the prior art known to those of ordinary skill in the art to which the present invention pertains.

### SUMMARY OF THE INVENTION

It is an object of the present invention to provide lipid nanoparticles, which confine the delivery of nucleic acid only to the site of administration upon topical administration, thus reducing systemic side effects, thereby increasing safety, and increasing the duration of effective expression at the site of administration, and a lipid nanoparticle composition comprising the lipid nanoparticles and a nucleic acid.

It is another object of the present invention to provide a vaccine comprising the lipid nanoparticle composition.

It is still another object of the present invention to provide a method of preventing or treating a disease comprising administering the lipid nanoparticle composition to a subject.

It is yet another object of the present invention to provide the use of the lipid nanoparticle composition for preventing or treating a disease.

It is still yet another object of the present invention to provide the use of the lipid nanoparticle composition for the manufacture of a medicament for preventing or treating a disease.

In order to accomplish the above objects, the present invention provides a lipid nanoparticle (LNP) comprising (A) an ionizable lipid; (B) cationic cholesterol; (C) cholesterol; (D) a helper lipid; and (E) a PEG lipid (polyethylene glycol lipid), in which the molar ratio of (B) cationic cholesterol to (C) cholesterol is 1:0.1 to 1:10.

In addition, the present invention provides a lipid nanoparticle composition comprising the lipid nanoparticle and a nucleic acid.

In addition, the present invention provides a vaccine comprising the lipid nanoparticle composition.

In addition, the present invention provides a method of preventing or treating a disease comprising administering the lipid nanoparticle composition to a subject.

In addition, the present invention provides the use of the lipid nanoparticle composition for preventing or treating a disease.

In addition, the present invention provides the use of the lipid nanoparticle composition for the manufacture of a medicament for preventing or treating a disease.

### BRIEF DESCRIPTION OF DRAWINGS

FIG. 1 shows the protein expression distribution after intramuscular injection of a lipid nanoparticle composition comprising cationic cholesterol according to an embodiment of the present invention in mice, along with a graph showing results of measurement of luminescence in liver tissue and muscle tissue.

FIG. 2 shows exemplary structures of cationic cholesterol of the present invention.

FIG. 3 shows exemplary structures of ionizable lipids evaluated in the present invention.

FIG. 4 shows the protein expression distribution after intramuscular injection to mice with lipid nanoparticles using 9 different ionizable lipids without/with cationic cholesterol.

FIG. 5 shows the protein expression level in liver and muscle tissues after intramuscular administration to mice at 6 hour and 7 days post-injection.

FIG. 6 shows the results of measuring the particle size and zeta potential of lipid nanoparticles used in an embodiment of the present invention.

FIG. 7 shows the distribution of protein expression after intramuscular injection in mice, depending on the change in the proportion of cationic cholesterol in lipid nanoparticles.

FIG. 8 is a graph showing the results of luminescence measurements of muscle tissue from FIG. 7.

FIG. 9 is a graph showing the results of luminescence measurement of liver tissue from FIG. 7.

FIG. 10 is a graph showing the luminescence ratio of muscle tissue to liver tissue from FIG. 7.

FIG. 11 shows the distribution of protein expression after intramuscular injection in mice, depending on the type of cationic cholesterol used in lipid nanoparticles.

FIG. 12 is a graph showing the results of luminescence measurement of muscle tissue from FIG. 11.

FIG. 13 is a graph showing the results of luminescence measurement of liver tissue from FIG. 11.

FIG. 14 is a graph showing the luminescence ratio of muscle tissue to liver tissue from FIG. 11.

FIG. 15 shows the protein expression levels in liver and muscle tissues at 6 hour and 7 days post-injection after intramuscular administration to mice, depending on the type of cationic cholesterol.

FIG. 16 shows antigen-specific antibody titers induced after two intramuscular injections of lipid nanoparticles without/with cationic cholesterol to mice, measured at 4, 6, 8, and 11 weeks post-first immunization.

FIG. 17 shows the protein expression kinetics after subcutaneous injection of lipid nanoparticles without/with cationic cholesterol to mice.

FIG. 18 shows the plasma concentration kinetics of erythropoietin in blood after intramuscular injection of lipid nanoparticles without/with cationic cholesterol to mice.

FIG. 19 shows the protein expression kinetics after intramuscular injection of lipid nanoparticles with cationic cholesterol or cationic lipid DOTAP to mice.

FIG. 20 shows the protein expression kinetics in mice after intramuscular injection of lipid nanoparticles with cationic cholesterol and self-amplifying mRNA.

### DETAILED DESCRIPTION AND PREFERRED EMBODIMENTS OF THE INVENTION

Unless otherwise defined, all technical and scientific terms used herein have the same meanings as typically understood by those skilled in the art to which the present invention belongs. In general, the nomenclature used herein is well known in the art and is typical.

In an embodiment of the present invention, it was expected to confine mRNA expression only to the site of administration and to maintain the administration dose by reducing the amount lost outside the site of administration. In addition, administration of a lipid nanoparticle composition of the present invention was confirmed to increase the duration of protein expression at the site of intramuscular or subcutaneous administration.

Accordingly, in an aspect, the present invention is directed to a lipid nanoparticle (LNP) comprising (A) an ionizable lipid; (B) cationic cholesterol; (C) cholesterol; (D) a helper lipid; and (E) a PEG lipid (polyethylene glycol lipid), in which the molar ratio of (B) cationic cholesterol to (C) cholesterol is 1:0.1 to 1:10.

In the lipid nanoparticles according to the present invention, the molar ratio of (B) cationic cholesterol to (C) cholesterol may be 1:0.1 to 1:10, preferably 1:0.2 to 1:5, more preferably 1:0.33 to 1:3, most preferably 1:1. Here, it was confirmed that protein expression in liver tissue decreased when the proportion of cationic cholesterol increased and also that protein expression in muscle tissue decreased when the proportion of cationic cholesterol exceeded a specific ratio.

In the lipid nanoparticles according to the present invention, the molar ratio of (B) cationic cholesterol to (A) ionizable lipid may be 1:0.5 to 1:20, preferably 1:1 to 1:10, more preferably 1:2 to 1:5, most preferably 1:2.59.

In the lipid nanoparticles according to the present invention, the molar ratio of (B) cationic cholesterol to (D) helper lipid may be 1:0.2 to 1:10, preferably 1:0.33 to 1:5, more preferably 1:0.5 to 1:2, most preferably 1:0.518 (1.93:1).

In the lipid nanoparticles according to the present invention, the molar ratio of (B) cationic cholesterol to (E) PEG lipid may be 1:0.01 to 1:1, preferably 1:0.02 to 1:0.2, more preferably 1:0.05 to 1:0.1, most preferably 1:0.078 (12.87:1).

In addition, the lipid nanoparticles according to the present invention preferably comprise 30 to 80 mol % of the ionizable lipid; 0.01 to 50 mol % of the cationic cholesterol; and 0.01 to 50 mol % of the cholesterol, more preferably 40 to 60 mol % of the ionizable lipid; 5 to 25 mol % of the cationic cholesterol; and 5 to 25 mol % of the cholesterol, most preferably 45 to 55 mol % of the ionizable lipid; 15 to 25 mol % of the cationic cholesterol; and 15 to 25 mol % of the cholesterol.

In addition, the lipid nanoparticles preferably further comprise 0.01 to 20 mol %, more preferably 5 to 15 mol %, most preferably 8 to 12 mol % of the helper lipid (phospholipid). In addition, the lipid nanoparticles preferably further comprise 0.01 to 10 mol %, more preferably 0.01 to 5 mol %, most preferably 1 to 2 mol % of the PEG lipid.

The lipid nanoparticles according to the present invention may have a zeta potential of 5 mV to 15 mV and a particle size (Z-average) of 50 nm to 250 nm. The particle size (Z-average) and zeta potential of the lipid nanoparticles were measured using a Zetasizer Pro (Malvern Instruments, United Kingdom). The particle size was measured after dilution using 1× DPBS, and 10 mM NaCl was used for zeta potential measurement. Based on results of measurement, the particle size was similar, but the composition comprising cationic cholesterol exhibited a higher zeta potential.

In the lipid nanoparticles according to the present invention, the cationic cholesterol may be, but is not limited to, at least one selected from the group consisting of AC-cholesterol (3β-[N-(aminoethane)carbamoyl]-cholesterol), MC-cholesterol (3β-[N-(N′-methylaminoethane)carbamoyl]-cholesterol), DC-cholesterol (3β-[N-(N′,N′-dimethylaminoethane)carbamoyl]-cholesterol), DMHAPC-cholesterol (3-[N-[3-[(2-hydroxyethyl)dimethylammonio]propyl]carbamate]), DMPAC-cholesterol (3-[[3-(dimethylamino)propyl]carbamate]), MHAPC-cholesterol (3-[N-[3-[ (2-hydroxyethyl)methylamino]propyl]carbamate]), HAPC-cholesterol (3-[N-[3-[(2-hydroxyethyl)amino]propyl]carbamate]), OH-cholesterol (N-[2-[(2-hydroxyethyl)amino]ethyl]-(3β)-cholest-5-ene-3-carboxamide), and OH-C-cholesterol (3-[N-[2-[ (2-hydroxyethyl)amino]ethyl]carbamate]).

In the lipid nanoparticles according to the present invention, the ionizable lipid may be, but is not limited to, at least one selected from the group consisting of DLin-DMA (1, 2-dilinoleyloxy-N, N-dimethylaminopropane), DLin-KC2-DMA (2,2-dilinoleyl-4-(2-dimethylaminoethyl)-[1, 3]-dioxolane), DLin-MC3-DMA ((6Z, 9Z, 28Z, 31Z)-heptatriaconta-6,9,28,31-tetraen-19-yl-4-(dimethylamino) butanoate), DODAP (1,2-dioleoyl-3-dimethylammonium propane), DODMA (N,N-dimethyl-(2,3-dioleyloxy)propylamine), cKK-E12 represented by Chemical Formula 1 below, C12-200 represented by Chemical Formula 2 below, ATX-002 represented by Chemical Formula 3below, and SM-102 represented by Chemical Formula 4 below.

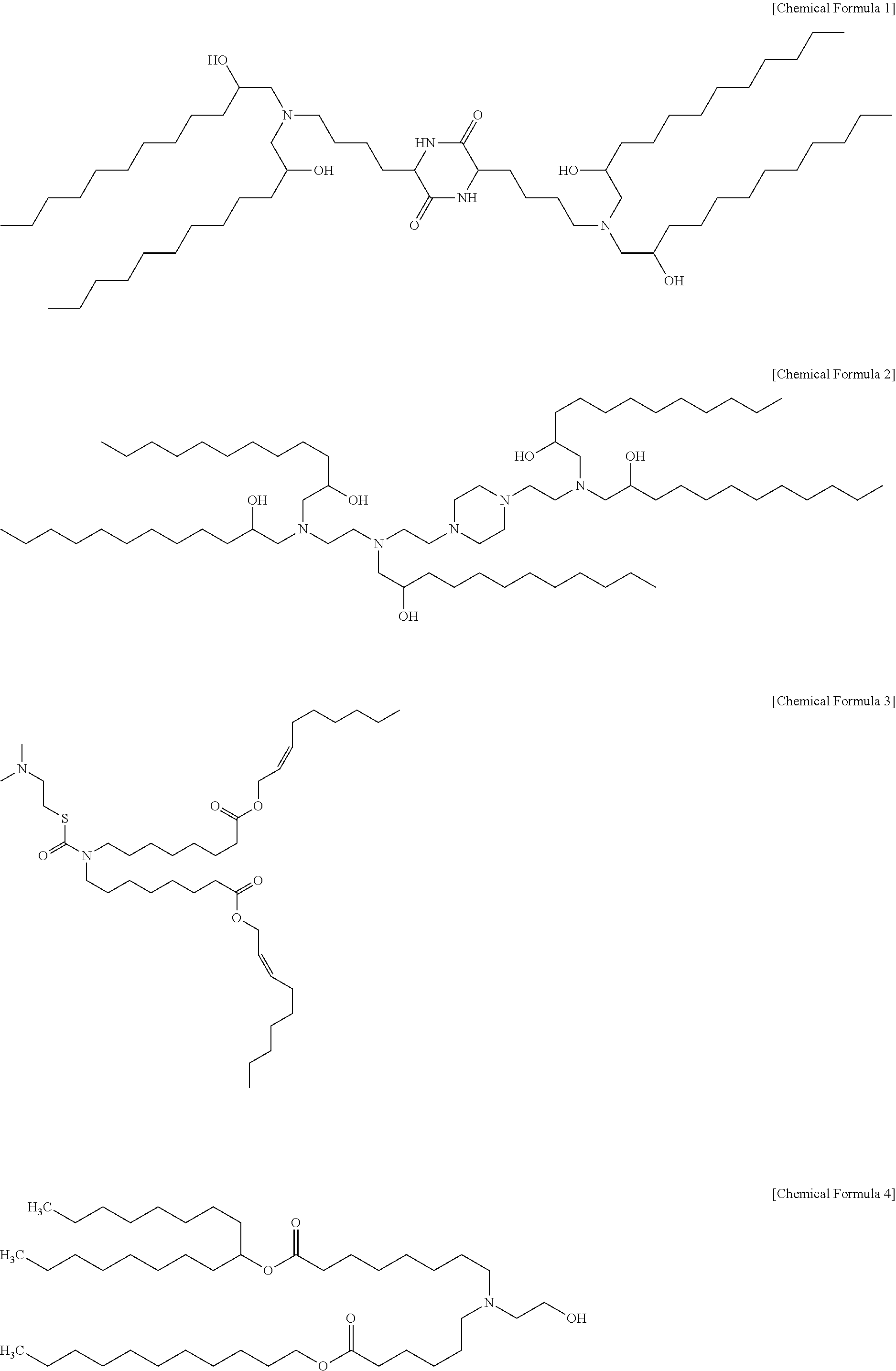

In addition, in the lipid nanoparticles according to the present invention, the ionizable lipid may be, but is not limited to, at least one selected from the group consisting of 1-linoleoyl-2-linoleyloxy-3-dimethylaminopropane (DLin-2-DMAP), 1,2-dilinoleyl carbamoyloxy-3-dimethylaminopropane (DLin-C-DAP), 1,2-dilinoleoyl-3-dimethylaminopropane (DLin-DAP), 2,2-dilinoleyl-4-dimethylaminomethyl-[1,3]-dioxolane (DLin-K-DMA), dioctadecylamidoglycylcarboxyspermine (DOGS), spermine cholesteryl carbamate (GL-67), bis-guanidinium-spermidine-cholesterol (BGTC), 1,1′-(2-(4-(2-((2-(bis(2-hydroxydecyl)amino)ethyl) (2-hydroxydecyl)amino)ethyl)piperazin-1-yl)ethylazandiyl)didodecan-2-ol (C12-200), N-t-butyl-N′-tetradecyl-amino-propionamidine (diC14-amidine), dimethyldioctadecylammonium bromide (DDAB), N-(1,2-dimyristyloxyprop-3-yl)-N,N-dimethyl-N-hydroxyethylammonium bromide (DMRIE), N, N-dioleyl-N, N-dimethylammonium chloride (DODAC), dioleyl oxypropyl-3-dimethylhydroxyethylammonium bromide (DORIE), N-(1-(2,3-dioleyloxy) propyl)-N-2-(sperminecarboxamido)ethyl)-N,N-dimethylammonium trifluoroacetate (DOSPA), 1, 2-dioleoyl trimethylammonium propane chloride (DOTAP), N-(1-(2, 3-dioleyloxy) propyl)-N,N,N-trimethylammonium chloride (DOTMA), and aminopropyl-dimethyl-bis(dodecyloxy)-propanaminium bromide (GAP-DLRIE).

In addition, in the lipid nanoparticles according to the present invention, the ionizable lipid is more preferably a lipid containing tertiary amine.

In the lipid nanoparticles according to the present invention, the helper lipid (phospholipid) may be, but is not limited to, at least one selected from the group consisting of DMPC (1,2-dimyristoyl-sn-glycero-3-phosphatidylcholine), DOPC (1,2-dioleoyl-sn-glycero-3-phosphocholine), DOPI (1,2-dioleoyl-sn-glycero-3-phospho-(1′-myo-inositol)), DOPE (1,2-dioleoyl-sn-glycero-3-phosphoethanolamine), DPPC (1,2-dipalmitoyl-sn-glycero-3-phosphocholine), DSPC (1,2-distearoyl-sn-glycero-3-phosphocholine), DSPI (1,2-distearoyl-sn-glycero-3-phosphoinositol), and DLPC (1,2-dilinoleoyl-sn-glycero-3-phosphocholine).

In addition, in the lipid nanoparticles according to the present invention, the helper lipid (phospholipid) may be, but is not limited to, at least one selected from the group consisting of 1,2-dilinoleoyl-sn-glycero-3-phosphocholine (DLPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1, 2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1, 2-diphytanoyl-sn-glycero-3-phosphoethanolamine (4ME 16:0 PE), 1,2-distearoyl-sn-glycero-3-phosphoethanolamine, 1,2-dilinoleoyl-sn-glycero-3-phosphoethanolamine, 1, 2-dilinolenoyl-sn-glycero-3-phosphoethanolamine, 1, 2-diarachidonoyl-sn-glycero-3-phosphoethanolamine, 1, 2-didocosahexaenoyl-sn-glycero-3-phosphoethanolamine, 1, 2-dioleoyl-sn-glycero-3-phospho-rac-(1-glycerol) sodium salt (DOPG), dipalmitoyl phosphatidylglycerol (DPPG), palmitoyl oleoyl phosphatidylethanolamine (POPE), distearoyl-phosphatidyl-ethanolamine (DSPE), dipalmitoyl phosphatidylethanolamine (DPPE), dimyristoyl phosphoethanolamine (DMPE), 1-stearoyl-2-oleoyl-phosphatidylethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyl oleoyl phosphatidylcholine, lysophosphatidylcholine, and lysophosphatidylethanolamine (LPE).

In the lipid nanoparticles according to the present invention, the PEG lipid may be, but is not limited to, at least one selected from the group consisting of PEG-modified phosphatidylethanolamine, PEG-modified phosphatidic acid, PEG-modified ceramide, PEG-modified dialkylamine, PEG-modified diacylglycerol, and PEG-modified dialkylglycerol.

In addition, the PEG lipid preferably comprises a PEG moiety having a size of 100 Da to 20 kDa, and is more preferably, but is not limited to, at least one selected from the group consisting of DMG-PEG2000 (1,2-dimyristoyl-rac-glycero-3-methoxypolyethylene glycol-2000), DSPE-PEG2000(1,2-distearoyl-sn-glycero-3-phosphoethanolamine-N-[amino(polyethylene glycol)-2000], and ceramide-PEG2000 (N-palmitoyl-sphingosine-1 -{succinyl[methoxy(polyethylene glycol))2000]}).

Another aspect, the present invention is directed to a lipid nanoparticle composition comprising the lipid nanoparticles and a nucleic acid.

In the lipid nanoparticle composition according to the present invention, the nucleic acid may be at least one selected from the group consisting of mRNA, SiRNA, aiRNA, miRNA, dsRNA, shRNA, IncRNA, saRNA, rRNA, RNA, DNA, CDNA, plasmid, aptamer, tRNA, piRNA, circRNA, antisense oligonucleotide, ribozyme, PNA, and DNAzyme, and is most preferably mRNA, but is not limited thereto.

The N/P ratio of the lipid nanoparticle composition according to the present invention is preferably 2 to 12, more preferably 4 to 8. The N/P ratio is determined by dividing N which is the number of moles of protonatable amine groups comprised in the lipid nanoparticle composition by P which is the number of moles of phosphate groups in mRNA.

Still another aspect, the present invention is directed to a vaccine comprising the lipid nanoparticle composition.

In the present invention, the term “vaccine” is understood as a prophylactic or therapeutic substance providing at least one antigen, preferably immunogen. The antigen or: immunogen may be derived from any substance suitable for vaccination. For example, an antigen or immunogen may be derived from a pathogen, such as a bacterial or viral particle, or a tumor or cancerous tissue. The antigen or immunogen stimulates the body's adaptive immune system that provides an adaptive immune response.

Yet another aspect, the present invention is directed to a method of preventing or treating a disease comprising administering the lipid nanoparticle composition to subject.

A further aspect, the present invention is directed to the use of the lipid nanoparticle composition for preventing or treating a disease.

Still a further aspect, the present invention is directed to the use of the lipid nanoparticle composition for the manufacture of a medicament for preventing or treating a disease.

As used herein, the term “preventing” refers to any action that prevents the onset of a disease or delays progression thereof by administration of the composition. In addition, the term “treating” refers to any action in which the symptoms of a disease are ameliorated or the symptoms are alleviated or eliminated by administration of the composition.

As used herein, the term “subject” refers to a mammal, preferably a human, suffering from or at risk of a condition or disease that may be alleviated, suppressed, or treated by administering the composition according to the present invention.

As used herein, the term “administering” refers to the action of introducing the composition of the present invention to a subject by any appropriate method, and the route of administration may include various oral or parenteral routes so long as a drug is able to reach the target tissue. Parenteral administration may be intramuscular (IM), intravenous (IV), subcutaneous (SC), intraperitoneal (IP), intratumoral (IT), intradermal (ID), or intracerebral injection, and the administration dose may vary depending on the status and weight of a patient, the severity of a disease, the type of drug, and the route and time of administration, but may be appropriately selected by those skilled in the art.

The administration dose of the composition of the present invention to the human body may vary depending on the patient's age, weight, gender, dosage form, health status, and severity of a disease.

In the present invention, when formulating the composition, diluents or excipients such as fillers, extenders, binders, wetting agents, disintegrants, surfactants, and the like may be typically used. Formulations for parenteral administration may include sterilized aqueous solutions, non-aqueous solvents, suspending agents, emulsions, lyophilized formulations, suppositories, and the like. Examples of non-aqueous solvents or suspending agents may include propylene glycol, polyethylene glycol, vegetable oils such as olive oil, injectable esters such as ethyl oleate, and the like. Examples of bases for suppositories may include Witepsol, Macrogol, Tween 61, cacao butter, laurin butter, glycerol, gelatin, and the like.

A better understanding of the present invention may be obtained through the following examples. These examples are merely set forth to illustrate the present invention, and are not to be construed as limiting the scope of the present invention, as will be apparent to those skilled in the art.

### Example 1: Preparation of Lipid Nanoparticles Comprising Cationic Cholesterol and Confirmation of Properties Thereof

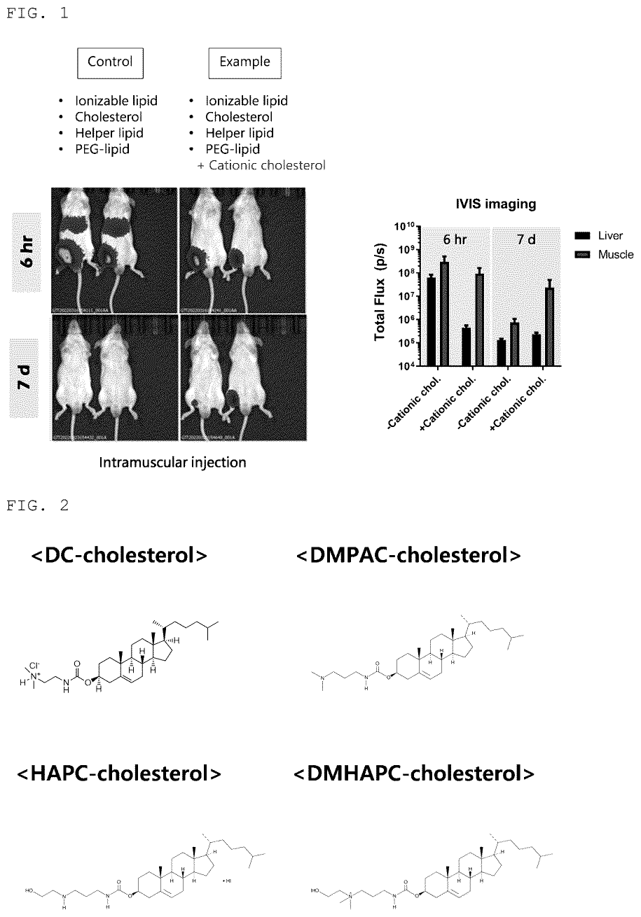

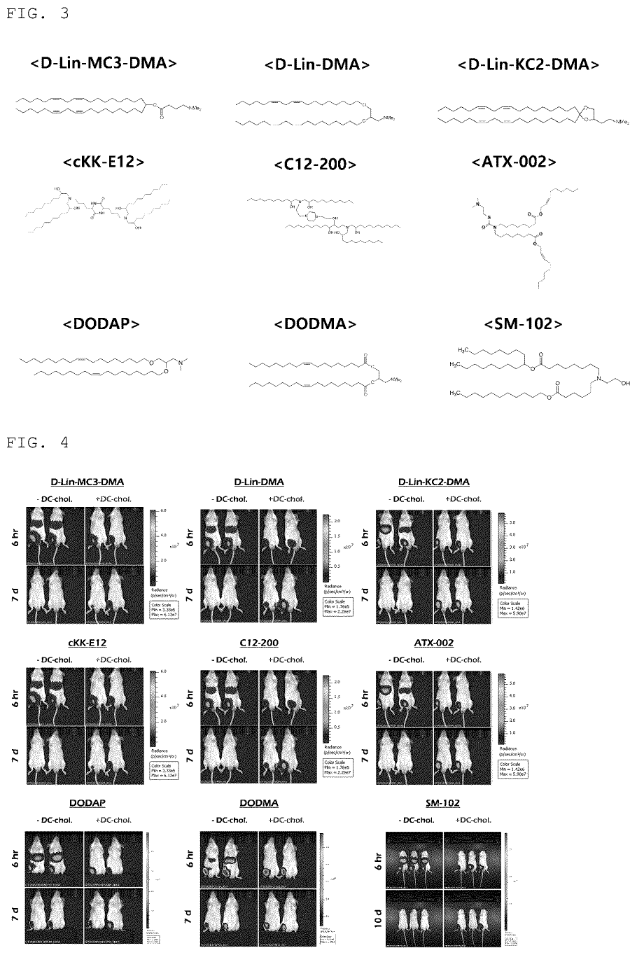

An ionizable lipid, cholesterol, cationic cholesterol, phospholipid, and PEG-lipid were dissolved in ethanol in a molar ratio of 50:19.25:19.25:10:1.5 and then mixed in a volume ratio of 1:3 with mRNA dissolved in citrate buffer (pH 4.0, 50 mM). For a control, an ionizable lipid, cholesterol, phospholipid, and PEG-lipid were used in a molar ratio of 50:38.5:10:1.5 without comprising cationic cholesterol (FIG. 1). The cationic cholesterol used in Example was DC-cholesterol (Avanti Polar Lipids), HAPC-cholesterol (GLPBIO, USA), DMPAC-cholesterol (GLPBIO, USA), or DMHAPC-cholesterol (GLPBIO, USA) (FIG. 2), the ionizable lipid used was D-Lin-MC3-DMA (MedChemExpress, USA), D-Lin-DMA (MedChemExpress, USA), D-Lin-KC2-DMA (MedChemExpress USA), CKK-E12 (Organix, USA), C12-200 (Organix, USA), ATX-002 (Organix, USA), DODAP (Avanti Polar Lipids, USA), DOMDA (Avanti Polar Lipids, USA), or SM-102 (Xiamen Sinopeg Biotech, China) (FIG. 3). In addition, the phospholipid used was 1,2-distearoyl-sn-glycero-3-phosphocholine (DSPC) (Avanti Polar Lipids, USA), and the PEG-lipid used was 1,2 -dimyristoyl-rac-glycero-3-methoxypolyethylene glycol-2000 (DMG-PEG2000) (Avanti Polar Lipids, USA). As mRNA, CleanCap® Firefly Luciferase mRNA (TriLink, USA), CleanCap® Erythropoietin mRNA (TriLink, USA), HA mRNA (in-house production), or self-amplifying mRNA (in-house production) was used.

For production of lipid nanoparticles, NanoAssemblr® Ignite™ (Precision Nanosystems, Inc. Canada) was used, and the total flow rate was set to 12 mL/min. The prepared lipid nanoparticles were subjected to ethanol removal, buffer exchange, and concentration using an Amicon® Ultra Centrifugal Filter, MWCO 10 kDa (Millipore, USA). 1× DPBS (Thermo Scientific, USA) was used for dilution and buffer exchange.

In order to analyze the properties of the formed lipid nanoparticles, the particle size (Z-average) and zeta potential were measured using a Zetasizer Pro (Malvern Instruments, United Kingdom). The particle size was measured after dilution using 1× DPBS, and 10 mM NaCl was used for zeta potential measurement. The results of measurement thereof are shown in Table 1 below. The amount of mRNA was measured using a Ribogreen RNA assay kit (Invitrogen, USA).

TABLE 1

| | Z-average | Zeta potential |
|---|---|---|
| Lipid nanoparticles | (d, nm) | (mV) |
| D-Lin-MC3-DMA | 97.8 ± 0.7 | 1.8 ± 0.5 |
| D-Lin-MC3-DMA + DC- | 96.9 ± 0.5 | 9.7 ± 0.6 |
| cholesterol | | |
| (cholesterol:DC- | | |
| cholesterol = 1:1) | | |
| D-Lin-DMA | 91.8 ± 1.2 | 7.6 ± 0.7 |
| D-Lin-DMA + DC-cholesterol | 102.2 ± 1.0 | 9.2 ± 1.0 |
| (cholesterol:DC- | | |
| cholesterol = 1:1) | | |
| D-Lin-KC2-DMA | 87.2 ± 1.0 | 6.6 ± 0.3 |
| D-Lin-KC2-DMA + DC- | 97.0 ± 1.6 | 8.9 ± 0.4 |
| cholesterol | | |
| (cholesterol:DC- | | |
| cholesterol = 1:1) | | |
| cKK-E12 | 133.0 ± 1.4 | −12.9 ± 0.4 |
| cKK-E12 + DC-cholesterol | 118.8 ± 0.3 | 9.3 ± 0.5 |
| (cholesterol:DC- | | |
| cholesterol = 1:1) | | |
| C12-200 | 130.7 ± 3.3 | 2.6 ± 0.1 |
| C12-200 + DC-cholesterol | 142.5 ± 2.2 | 8.4 ± 0.5 |
| (cholesterol:DC- | | |
| cholesterol = 1:1) | | |
| ATX-002 | 124.7 ± 1.8 | 3.9 ± 0.7 |
| ATX-002 + DC-cholesterol | 126.8 ± 0.2 | 10.7 ± 0.8 |
| (cholesterol:DC- | | |
| cholesterol = 1:1) | | |
| DODAP | 102.7 ± 1.3 | −3.5 ± 0.7 |
| DODAP + DC-cholesterol | 226.2 ± 3.1 | 10.2 ± 1.4 |
| (cholesterol:DC- | | |
| cholesterol = 1:1) | | |
| DODMA | 120.7 ± 0.4 | 3.7 ± 0.4 |
| DODMA + DC-cholesterol | 149.0 ± 1.0 | 9.4 ± 0.8 |
| (cholesterol:DC- | | |
| cholesterol = 1:1) | | |
| SM-102 | 88.1 ± 1.9 | 3.0 ± 0.2 |
| SM-102 + DC-cholesterol | 102.6 ± 0.4 | 13.1 ± 0.6 |
| (cholesterol:DC- | | |
| cholesterol = 1:1) | | |

For lipid nanoparticles formed by addition of cationic cholesterol, the particle size was similar and the cationic zeta potential was increased.

### Example 2: Evaluation of Drug Delivery Distribution and Duration After Intramuscular Injection of Lipid Nanoparticles Comprising Cationic Cholesterol

In order to evaluate drug delivery distribution after topical administration of the substance prepared in Example 1, lipid nanoparticles corresponding to 0.25 mg/kg mRNA were intramuscularly injected to the thigh of Balb/c mice (male, 5 weeks old) (injection volume: 50 μL). 6 hours or 7 days after injection, 150 mg/kg of D-luciferin (Perkin Elmer, USA) was intraperitoneally administered thereto, and after 15 minutes, bioluminescence was measured using an IVIS Lumina XR (Perkin Elmer, USA).

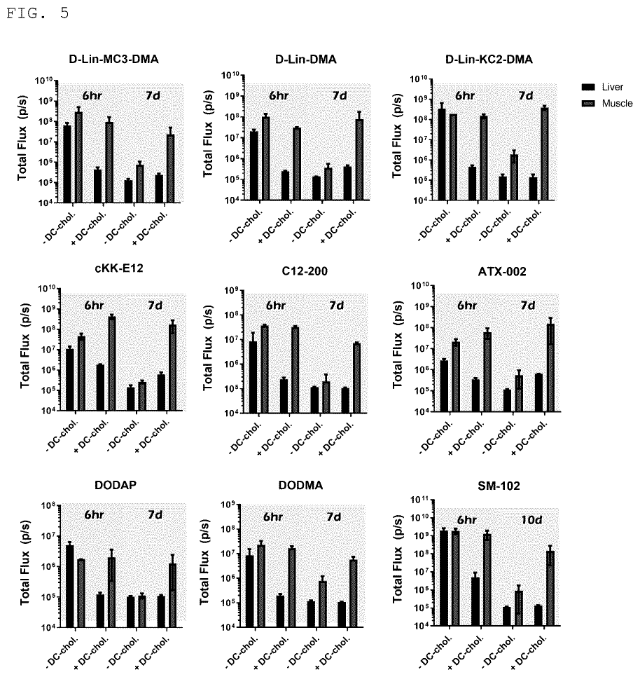

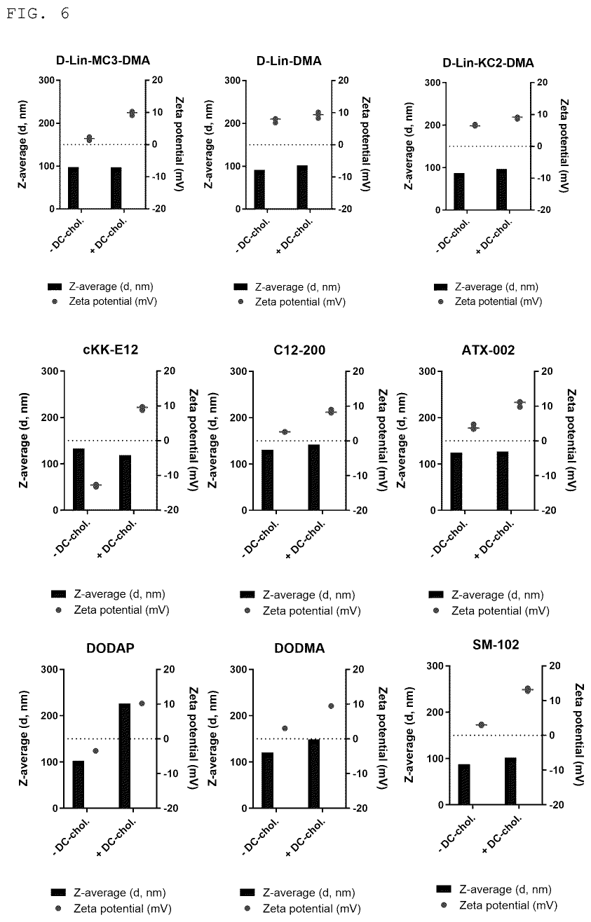

Luminescence levels of the administered muscle tissue and liver tissue were measured using whole-body images, and the results of drug delivery distribution after intramuscular injection of lipid nanoparticles using 9 ionizable lipids without/with cationic cholesterol to mice are shown in FIG. 4. Specifically, lipid nanoparticles using 9 ionizable lipids with luciferase mRNA encapsulated (D-Lin-MC3-DMA, D-Lin-DMA, D-Lin-KC2-DMA, CKK-E12, C12-200, ATX-002, DODAP, DODMA, SM-102) were injected intramuscularly to mice, and after 6 hours or 7 days, luminescence images were measured to evaluate the distribution and kinetics of protein expression. The luminescence signals in muscle and liver tissues at 6 hour and 7 days (FIG. 5) and the physical properties of lipid nanoparticles are presented in graphs (FIG. 6). As shown in FIG. 5, for conventional lipid nanoparticles without cationic cholesterol, a substantial amount of protein expression was seen in the liver, in addition to the administration site. However, when cationic cholesterol was included, protein expression was confined to muscle tissue. Moreover, at Day 7 post-injection, protein expression by the conventional lipid nanoparticles was greatly reduced, whereas the lipid nanoparticles with cationic cholesterol continuously maintained a significant level of protein expression.

The above results were obtained regardless of the type of ionizable lipid tested, which suggests that the composition ratio can be generally applied to lipid nanoparticles using an ionizable lipid.

### Example 3: Evaluation of Drug Delivery Distribution After Intramuscular Injection of Lipid Nanoparticles Depending on Proportion of Cationic Cholesterol

In order to identify an optimal composition of cationic cholesterol, protein expression profiles were evaluated with varying proportions of cationic cholesterol, as shown Table 2. The ionizable lipid used was D-Lin-MC3-DMA.

TABLE 2

| Lipid | | | | | | |
|---|---|---|---|---|---|---|
| nanoparticle | | | | | | |
| composition | A | B | C | D | E | F |
| Ionizable lipid | 50% | | | | | |
| Cationic | — | 3.9% | 9.6% | 19.3% | 28.9% | 38.5% |
| cholesterol | | | | | | |
| Cholesterol | 38.5% | 34.7% | 28.9% | 19.3% | 9.6% | — |

| | |
|---|---|
| Phospholipid | 10% |
| PEG-lipid | 1.5% |

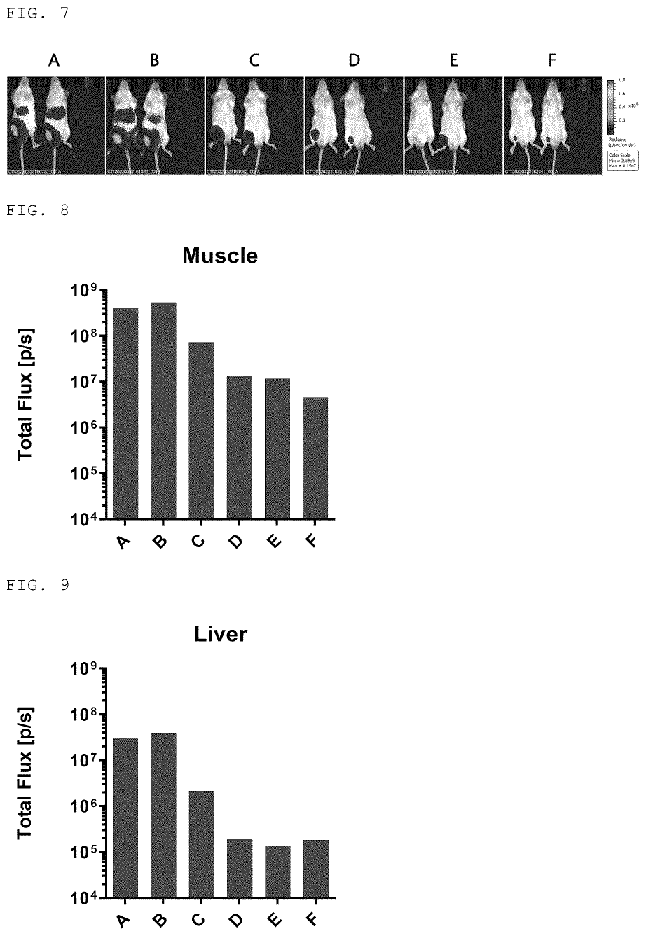

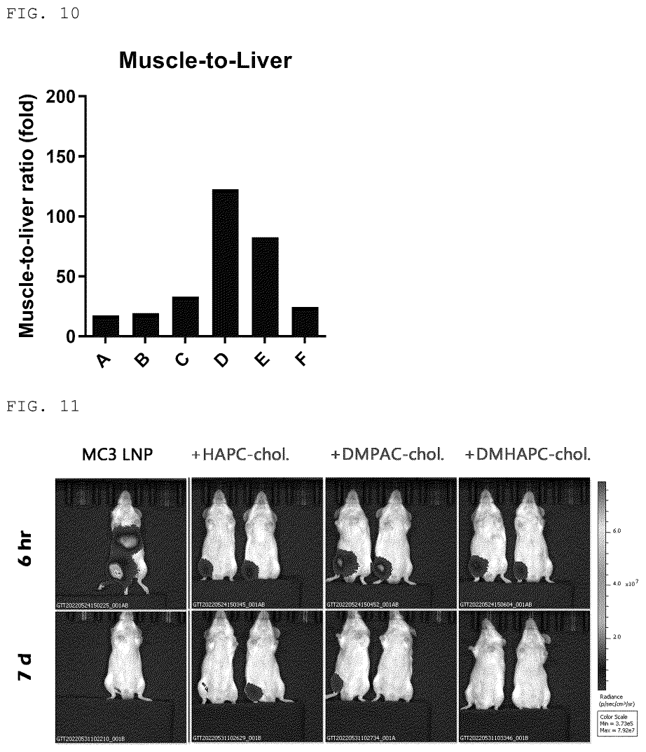

6 hours after intramuscular injection (0.25 mg/kg mRNA) to the thigh of Balb/c mice (male, 5 weeks old), bioluminescence was measured using an IVIS Lumina XR, and the results thereof are shown in FIG. 7. Specifically, lipid nanoparticles using D-Lin-MC3-DMA containing luciferase mRNA were encapsulated by comprising different proportions of cationic cholesterol, and luminescence images were measured 6 hours after intramuscular injection thereof to mice. The results of measurement of luminescence in the administered muscle tissue are shown in FIG. 8, the results of measurement of luminescence of liver tissue are shown in FIG. 9, and the luminescence ratio of muscle tissue to liver tissue is shown in FIG. 10. It was confirmed that the protein expression in liver tissue decreased with an increase in the proportion of cationic cholesterol and also that the protein expression in muscle tissue decreased when the proportion of cationic cholesterol exceeded a specific ratio (D). Therefore, the composition ratio capable of maintaining protein expression levels in muscle tissue and minimizing systemic delivery was determined (D; cholesterol: cationic cholesterol=1:1).

### Example 4: Evaluation of Drug Delivery Distribution After Intramuscular Injection of Lipid Nanoparticles Depending on Type of Cationic Cholesterol

In order to confirm drug delivery distribution after intramuscular injection depending on the type of cationic cholesterol, lipid nanoparticles were prepared and evaluated using the composition ratio selected in Example 3 (ionizable lipid: cationic cholesterol: cholesterol: phospholipid: PEG-lipid=50:19.3:19.3:10:1.5 molar ratio). Lipid nanoparticles using D-Lin-MC3-DMA containing luciferase mRNA were prepared by comprising different types of cationic cholesterol.

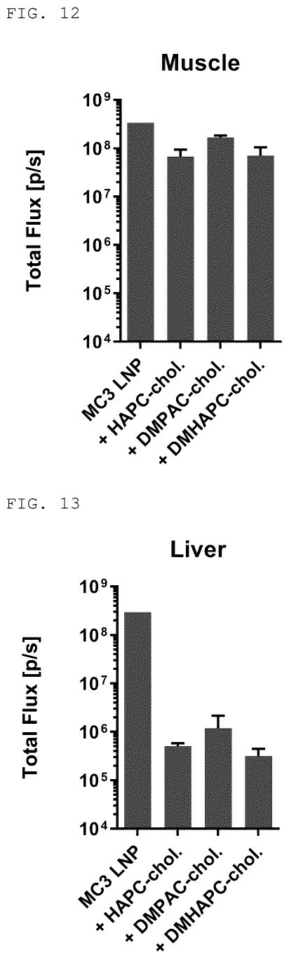

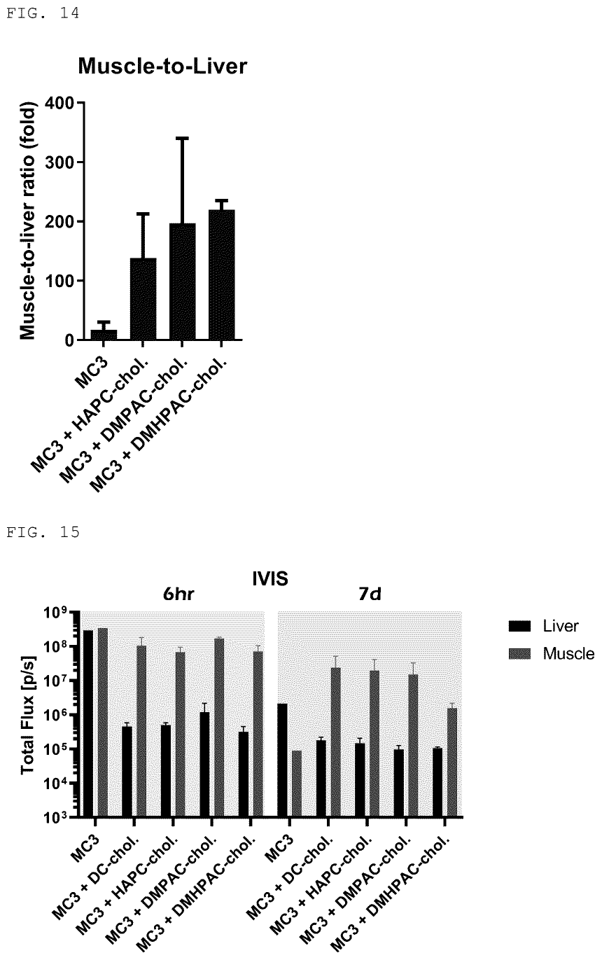

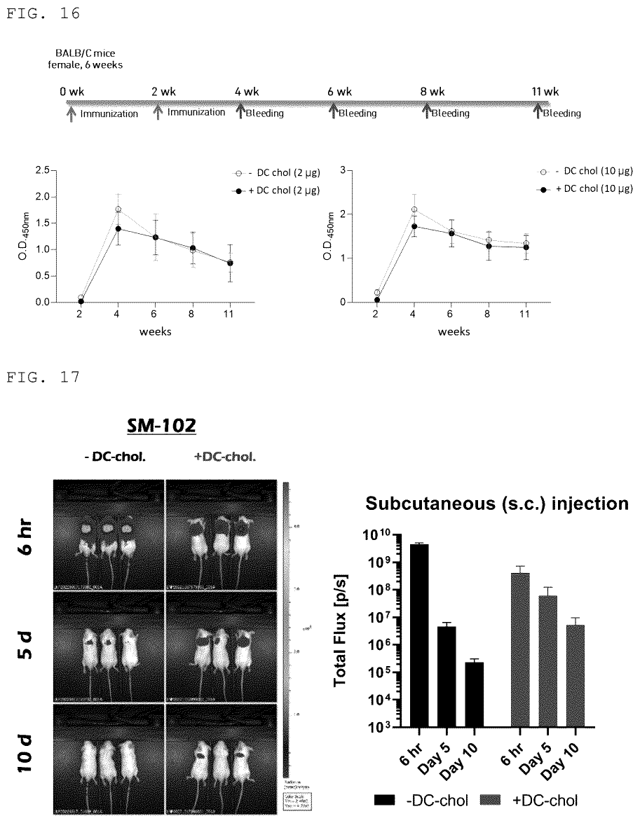

6 hours or 7 days after intramuscular injection (0.25 mg/kg mRNA) to the thigh of Balb/c mice (male, 5 weeks old), bioluminescence was measured using an IVIS Lumina XR, and the results thereof are shown in FIG. 11. Specifically, the results of measurement of luminescence in the administered muscle tissue are shown in FIG. 12, the results of measurement of luminescence of liver tissue are shown in FIG. 13, the luminescence ratio of muscle tissue to liver tissue is shown in FIG. 14, and the results of measurement of protein expression levels in muscle tissue and liver tissue 6 hours and 7 days after injection are shown in FIG. 15. The protein expression in liver tissue was confirmed to decrease due to inclusion of cationic cholesterol regardless of the type of cationic cholesterol evaluated (HAPC-cholesterol, DMPAC-cholesterol, DMHAPC-cholesterol).

### Example 5: Application of Flu Vaccine to Intramuscular Injection (IM) Route

In order to confirm the applicability of lipid nanoparticles comprising cationic cholesterol to vaccines, lipid nanoparticles including influenza HA mRNA and lipid nanoparticles comprising cationic cholesterol were prepared. Here, the ionizable lipid used was MC3. LNPs comprising 2 μg or 10 μg of HA mRNA were intramuscularly injected to the right thigh of Balb/c mice (female, 6 weeks old) twice at 2-week intervals using an insulin syringe. Serum was obtained through retro-orbital blood sampling at 2, 4, 6, and 9 weeks after second immunization, and HA antigen-specific IgG levels were measured by ELISA to confirm antigen-specific immunogenicity. As a result, at both 2 μg and 10 μg, it was confirmed that the response induced by the lipid nanoparticles comprising cationic cholesterol was similar to that induced by the lipid nanoparticles (FIG. 16).

### Example 6: Confirmation of Applicability to Subcutaneous Injection (SC) Route

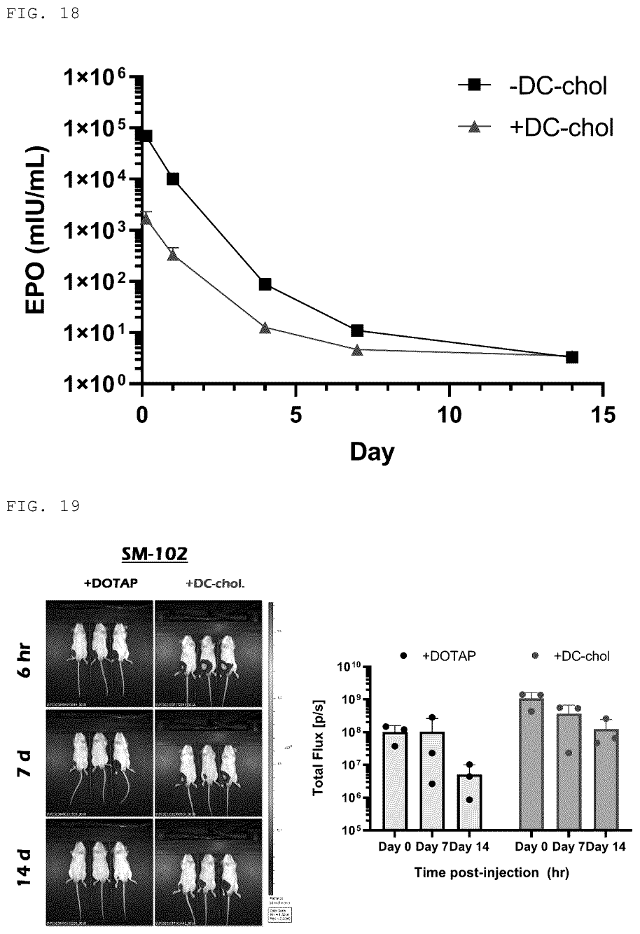

In order to evaluate drug delivery distribution after subcutaneous injection of SM-102 LNP among the substances prepared in Example 1, lipid nanoparticles corresponding to 0.25 mg/kg mRNA were subcutaneously (s.c.) injected to the napes of Balb/c mice (male, 6 weeks old) (injection volume: 50 μL). 6 hours, 5 days, 10 days, or 21 days after injection, 150 mg/kg of D-luciferin (Perkin Elmer, USA) was intraperitoneally administered thereto, and after 15 minutes, bioluminescence was measured using an IVIS Lumina XR (Perkin Elmer, USA). The results of measurement of images and luminescence values of the site of administration are shown in FIG. 17. It was confirmed that the duration of protein expression was maintained continuously up to 10 days when using lipid nanoparticles comprising cationic cholesterol compared to when using conventional lipid nanoparticles, even upon subcutaneous injection, like the results after intramuscular injection in Example 2.

### Example 7: Confirmation of Minimization of Systemic Delivery of Lipid Nanoparticles Comprising Cationic Cholesterol

In order to evaluate systemic exposed protein levels after intramuscular injection of SM-102 LNP among the substances prepared in Example 1, lipid nanoparticles corresponding to 2 mg/kg mRNA (erythropoietin mRNA) were intramuscularly (i.m.) injected to Balb/c mice (male, 6 weeks old) (injection volume: 50 μL). Blood was collected at 3 hours, 1 day, 4 days, 7 days, and 14 days after injection, and the concentration of serum erythropoietin was measured. The results of measurement of blood EPO concentration with time are shown in FIG. 18. The amount of protein in the blood was observed to be less than about 10 times when using lipid nanoparticles comprising cationic cholesterol compared to when using conventional lipid nanoparticles, indicating that the amount of systemic exposed protein can be reduced when cationic cholesterol is comprised.

### Example 8: Comparison of Effects Upon Application of Cationic Substance Other Than Cholesterol

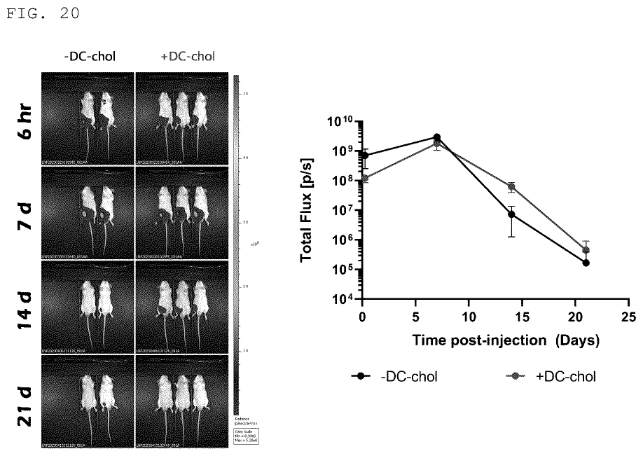

In order to compare effects with cationic lipid, other than cationic cholesterol, lipid nanoparticles were prepared and evaluated using the composition ratio selected in Example 3 (ionizable lipid: cationic cholesterol: cholesterol: phospholipid: PEG-lipid=50:19.3:19.3:10:1.5 molar ratio). Here, the cationic lipid used was DOTAP. In order to evaluate the distribution of drug delivery after intramuscular injection, lipid nanoparticles corresponding to 0.25 mg/kg mRNA were intramuscularly injected (0.25 mg/kg mRNA) to the thigh of Balb/c mice (male, 6 weeks old), and after 6 hours, 7 days, and 14 days, bioluminescence was measured using an IVIS Lumina XR, and the results thereof are shown in FIG. 19. Even when DOTAP was used instead of cationic cholesterol, the distribution of protein expression was similarly confined to the site of administration, but the duration of protein expression was relatively reduced at the site of administration (e.g. about 7 days for DOTAP vs. about 14 days for cationic cholesterol). Therefore, the cholesterol-based cationic substance was demonstrated to be essential for the lipid nanoparticles according to the present invention.

### Example 9: Confirmation of Applicability to Self-Amplifying RNA

In order to evaluate the applicability of SM-102 LNPs among the substances prepared in Example 1 to self-amplifying RNA (saRNA), lipid nanoparticles corresponding to 0.1 mg/kg mRNA were intramuscularly (i.m.) injected to Balb/c mice (male, 6 weeks old) (injection volume: 50 μL). 6 hours, 7days, 14 days, and 21 days after injection, bioluminescence was measured using an IVIS Lumina XR (Perkin Elmer, USA). The results of measurement of images and luminescence values of the site of administration are shown in FIG. 20. It was confirmed that the duration of protein expression was increased when using lipid nanoparticles comprising cationic cholesterol compared to when using conventional lipid nanoparticles, even upon application to saRNA, like the results of application to conventional mRNA (Example 1).

### INDUSTRIAL APPLICABILITY

According to the present invention, lipid nanoparticles for nucleic acid delivery are effective at minimizing systemic delivery of a drug upon topical administration and delivering the drug only to the site of administration. This reduces the amount of drug lost outside the site of administration, maintaining the administration dose at target site. In addition, the duration of protein expression at the muscle site can be increased upon topical administration of the lipid nanoparticles according to the present invention, which potentially lowers the therapeutic dose.

Although specific embodiments of the present invention have been disclosed in detail above, it will be obvious to those skilled in the art that the description is merely of preferable exemplary embodiments and is not to be construed as limiting the scope of the present invention. Therefore, the substantial scope of the present invention will be defined by the appended claims and equivalents thereto.

1. A lipid nanoparticle (LNP), comprising:
(A) an ionizable lipid;
(B) cationic cholesterol;
(C) cholesterol;
(D) a helper lipid; and
(E) a PEG lipid (polyethylene glycol lipid),
wherein a molar ratio of (B) cationic cholesterol to (C) cholesterol is 1:0.1 to 1:10.

2. The lipid nanoparticle according to claim 1, wherein a molar ratio of (B) cationic cholesterol to (A) ionizable lipid is 1:0.5 to 1:20.

3. The lipid nanoparticle according to claim 1, wherein a molar ratio of (B) cationic cholesterol to (D) helper lipid is 1:0.2 to 1:10.

4. The lipid nanoparticle according to claim 1, wherein a molar ratio of (B) cationic cholesterol to (E) PEG lipid is 1:0.01 to 1:1.

5. The lipid nanoparticle according to claim 1, wherein the lipid nanoparticle comprises 30 to 80 mol % of the ionizable lipid; 0.01 to 50 mol % of the cationic cholesterol; and 0.01 to 50 mol % of the cholesterol.

6. The lipid nanoparticle according to claim 5, wherein the lipid nanoparticle further comprises 0.01 to 20 mol % of the helper lipid.

7. The lipid nanoparticle according to claim 5, wherein the lipid nanoparticle further comprises 0.01 to 10 mol % of the PEG lipid.

8. The lipid nanoparticle according to claim 1, wherein the lipid nanoparticle has a zeta potential of 5 mV to 15 mV.

9. The lipid nanoparticle according to claim 1, wherein the lipid nanoparticle has a particle size (Z-average) of 50 nm to 250 nm.

10. The lipid nanoparticle according to claim 1, wherein the cationic cholesterol is at least one selected from the group consisting of AC-cholesterol (3β-[N-(aminoethane)carbamoyl]-cholesterol), MC-cholesterol (3β-[N-(N′-methylaminoethane) carbamoyl]-cholesterol), DC-cholesterol (3β-[N-(N′,N′-dimethylaminoethane)carbamoyl]-cholesterol), DMHAPC-cholesterol (3-[N-[3-[(2-hydroxyethyl)dimethylammonio]propyl]carbamate]), DMPAC-cholesterol (3- [[3-(dimethylamino)propyl]carbamate]), MHAPC-cholesterol (3-[N-[3-[(2-hydroxyethyl)methylamino]propyl]carbamate]), HAPC-cholesterol (3-[N-[3-[(2-hydroxyethyl)amino]propyl]carbamate]), OH-cholesterol (N-[2-[(2-hydroxyethyl)amino]ethyl]-(3β)-cholest-5-ene-3-carboxamide), and OH-C-cholesterol (3-[N-[2-[(2-hydroxyethyl)amino]ethyl]carbamate]).

11. The lipid nanoparticle according to claim 1, wherein the ionizable lipid is at least one selected from the group consisting of DLin-DMA (1,2-dilinoleyloxy-N,N-dimethylaminopropane), DLin-KC2-DMA (2,2-dilinoleyl-4-(2-dimethylaminoethyl)-[1,3]-dioxolane), DLin-MC3-DMA ((6Z,9Z,28Z,31Z)-heptatriaconta-6,9,28,31-tetraen-19-yl-4-(dimethylamino)butanoate), DODAP (1,2-dioleoyl-3-dimethylammonium propane), DODMA (N,N-dimethyl-(2,3-dioleyloxy) propylamine), cKK-E12 represented by Chemical Formula 1 below, C12-200 represented by Chemical Formula 2 below, ATX-002 represented by Chemical Formula 3 below, and SM-102 represented by Chemical Formula 4 below:

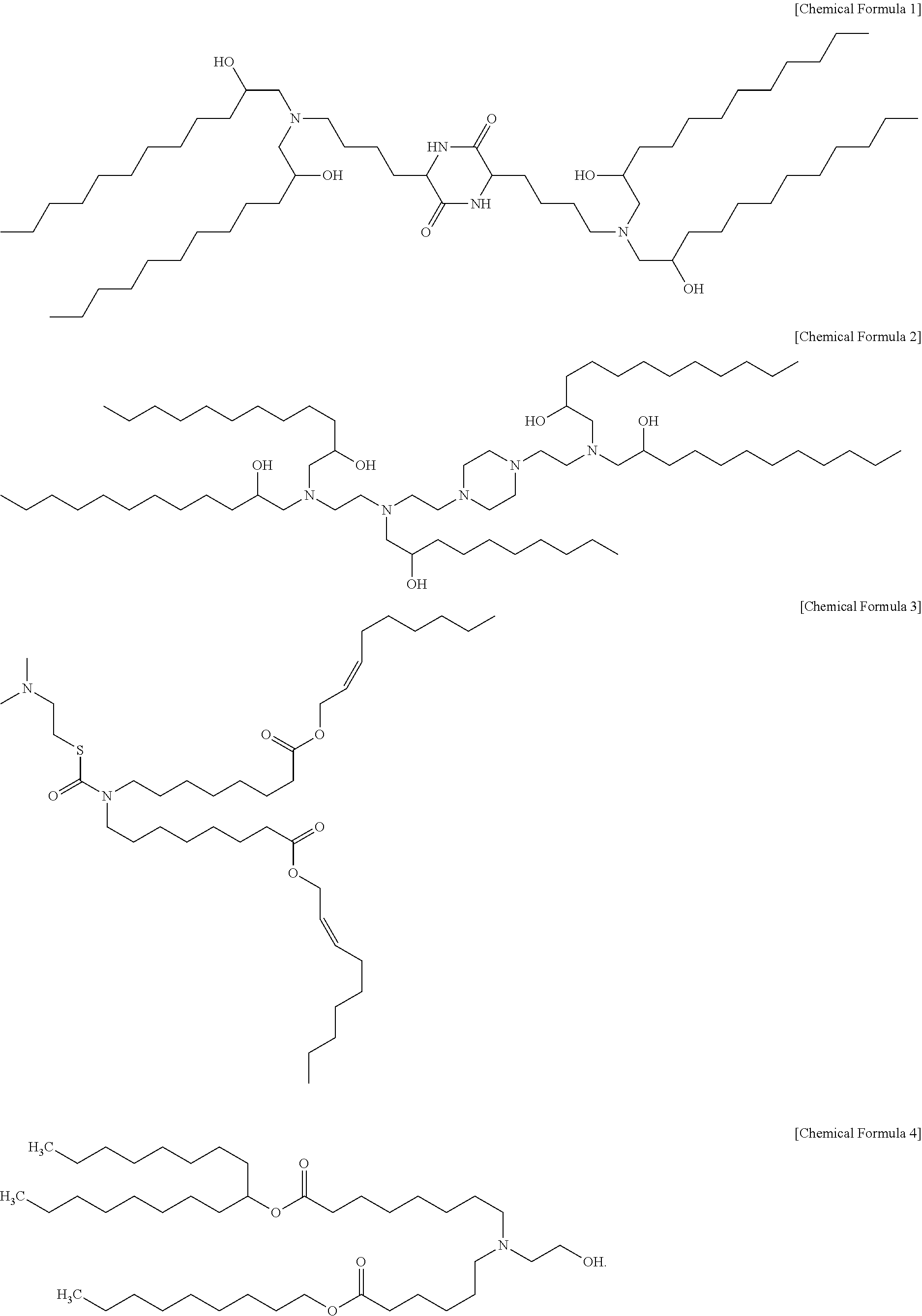

12. The lipid nanoparticle according to claim 1, wherein the helper lipid is at least one selected from the group consisting of DMPC (1,2-dimyristoyl-sn-glycero-3-phosphatidylcholine), DOPC (1,2-dioleoyl-sn-glycero-3-phosphocholine), DOPI (1,2-dioleoyl-sn-glycero-3-phospho-(1′-myo-inositol)), DOPE (1,2-dioleoyl-sn-glycero-3-phosphoethanolamine), DPPC (1,2-dipalmitoyl-sn-glycero-3-phosphocholine), DSPC (1,2-distearoyl-sn-glycero-3-phosphocholine), DSPI (1,2-distearoyl-sn-glycero-3-phosphoinositol), and DLPC (1,2-dilinoleoyl-sn-glycero-3-phosphocholine).

13. The lipid nanoparticle according to claim 1, wherein the PEG lipid is at least one selected from the group consisting of PEG-modified phosphatidylethanolamine, PEG-modified phosphatidic acid, PEG-modified ceramide, PEG-modified dialkylamine, PEG-modified diacylglycerol, and PEG-modified dialkyl glycerol.

14. A lipid nanoparticle composition comprising the lipid nanoparticle according to claim 1.

15. The lipid nanoparticle composition according to claim 14, wherein the nucleic acid is at least one selected from the group consisting of mRNA, siRNA, aiRNA, miRNA, dsRNA, shRNA, IncRNA, saRNA, rRNA, RNA, DNA, cDNA, plasmid, aptamer, tRNA, piRNA, circRNA, antisense oligonucleotide, ribozyme, PNA, and DNAzyme.

16. The lipid nanoparticle composition according to claim 14, wherein the composition has an N/P ratio of 2 to 12.

17. A vaccine comprising the lipid nanoparticle composition according to claim 14.

18. A method of preventing or treating a disease comprising administering the lipid nanoparticle composition according to claim 14 to a subject.
